(19) United States
(12) Patent Application Publication
(10) Pub. No.: US 2006/0156440 A1
(43) Pub. Date: Jul. 13, 2006

(54) Method for enhancing the nutritive value of plant extract

(75) Inventors: Dominique Michaud, Quebec (CA); Daniel Rivard, Quebec (CA); Raphael Anguenot, Quebec (CA); Sonia Trepanier, Quebec (CA); Louis-Philippe Vezina, Quebec (CA); France Brunelle, Quebec (CA)

Correspondence Address: OGILVY RENAULT LLP, 1981 MCGILL COLLEGE AVENUE, SUITE 1600, MONTREAL, QC, H3A2Y3, CA

(73) Assignee: UNIVERSITE LAVAL, Quebec (CA)

(21) Appl. No.: 10/519,845

(22) PCT Filed: Jul. 29, 2003
(86) PCT No.: PCT/CA03/01146

§ 371 Date: Aug. 4, 2005

Related U.S. Application Data
(60) Provisional application No. 60/398,783, filed on Jul. 29, 2002.

(51) Int. Cl. A01H 1/00; C12N 9/99; C12N 15/82; A61K 36/18

(52) U.S. Cl. 800/288; 435/184

(57) ABSTRACT

The present invention relates to a method for increasing the stability of endogenous proteins recovered from plant cells or plants. The preservation of endogenous proteins integrity of endogenous protein occurs by neutralizing proteolysis in crude extracts, particularly by the use of genetic alteration of plant cells or plants that express recombinant protease inhibitors or altered activity of specific target proteases.

### TECHNICAL FIELD

The present invention relates to a method for increasing the nutritive value of a plant extract, by inhibiting the degradation of its endogenous protein content. The method uses genetic alteration of plants to reduce protease-mediated degradation of endogenous proteins.

### BACKGROUND ART

Plants are well recognized as an excellent source of nutritive ingredients useful for human health as well as animal feeding. Methods to improve the nutritional value of plant forage crops for animal feeding are already documented and some are described in the following paragraphs.

One approach to improve the nutritive value of forage crop is to optimise their amino acid balance. This may be done by introducing into these plants, genes encoding proteins high in methionine driven, by a strong constitutive promoter or a leaf promoter. In order to significantly alter the amino acid balance of legume forages, the foreign proteins should contain about 15 to 25% of S-amino acids, and constitute 5 to 10% of the total leaf protein. To achieve such levels of protein accumulation, one has to ensure not only maximum levels of transcription and translation of the gene, but also the stability of the protein.

Most of the concerted efforts in regard to the nutritional improvements of plants have focused on seed proteins. Since corn and other cereal crops are not easily transformable, most work directed to seed protein modification has involved testing the stability of modified prolamine proteins in transgenic tobacco. The synthesis of lysine containing alpha zeins was also analyzed in transgenic tobacco and petunia seeds (Williamson et al., 1988). Both the normal and modified proteins were found to have a very short half-life.

One approach to increase the pools of particular amino acids in plants has been to introduce bacterial genes encoding for key regulatory enzymes in amino acid biosynthetic pathways in plants. A bacterial gene encoding for anaspartate kinase which is desensitised to feedback inhibition by lysine and threonine was fused to the beta-phaseolin gene promoter and introduced into tobacco. The seeds of the transgenic tobacco showed increased levels of free threonine and methionine (Galili, 1995, Plant Cell. 7:899-906).

Although methods for improving the amino acid content of plants have been developed, very little effort has been made with regards to improving forage crop protein quality. For example, Schoeder et al., (199, Aust. J. Plant Physiol. 18:495-505) introduced the chicken ovalbumin gene (cDNA), driven by a CaMV 35S promoter, into alfalfa. The transgenic alfalfa plants, however, showed very low level accumulation of protein in the leaves (0.005%). The reason explaining for such low abundance of this protein in transgenic alfalfa leaves was not determined, but this obsevation shows that the introduction of an exogenous high value protein in plants may not be necessarily a suitable method to improve the nutritive value of plants, presumably because of the low level of expression.

The nutritive value of a plant extract is in part dependent of the quality of its endogenous proteins. As for recombinant proteins produced in plants, the yields of stable endogenous proteins are closely associated with the stability of proteins during their accumulation in plants and during the processing of plant extracts.

One source of the low yield of valuable protein production is the proteolytic activity of endogenous proteases that degrade proteins. Plant cells are known to possess several poorly-specific proteases. Leaf vacuolar proteases active in the mildly-acidic pH range, in particular, may significantly alter the stability of many proteins and decrease the nutritive value of plant extracts.

Indeed, plant proteases may degrade endogenous proteins during two critical steps during the production of nutritive extracts from plants. The degradation may occurs 1) in planta, during the accumulation of the proteins, and 2) ex planta, at the time of cell disruption during the processing of plant extracts. The latter may be of major importance, since atduring this step, cell disruption liberates a pool of proteases from all parts and cell compartments of the plants. The basic process for producing nutritive extracts from plant leaves generally begins with disintegrating plant biomass and pressing the resulting pulp to produce a green juice. The green juice typically contains various proteins including proteases and a green pigmented material. It is of no use to process plants containing high levels of protein if these levels during and after the process are comparatively low. This invention focuses on the prevention of proteolysis occurring ex planta at the time of cell disruption, during the processing of plant extracts.

Little is known about interactions between plant proteases and their inhibitors and, yet there are no available plant lines harboring a low proteolytic phenotype. An actual way to overcome the proteolysis problem in plants is the targeting of recombinant proteins to organelles using appropriate targeting signals and their consequent accumulation in specific subcellular compartments. Because this strategy requires the modification of original protein sequences, it is not applicable to endogenous proteins.

Another way to reduce the level of protein hydrolysis in plant extracts is to “cool” (freeze) the plant materials following cutting. There is a considerable risk of proteolysis of endogenous proteins at the time of harvesting (Michaud and Asselin, 1995, J. Chromatography A698:263-279). During the harvesting and preparation of alfalfa pellets, for example, plant cells are disrupted and then release various compounds into the medium, including proteases that may alter the integrity of endogenous valuable proteins.

This degradation problem can also be resolved by the inclusion of low-molecular weight protease inhibitors in the extraction buffer (Michaud, 1998, Anal. Chimica Acta 372:173-185). However, although this strategy is useful in a small-scale production level, it is not suitable for the industrial scale, as well as for the food and feed markets, where proteins are produced in large amounts.

It is known from the art that expression of a recombinant protease inhibitor in plant may negatively affect the normal development of the transgenic plant as proteases participate in various metabolic events. It is also known in the art that recombinant protease inhibitors may be inserted in a plant in order to modify the metabolism of the plant to gain desirable agronomic characteristics. Moreover, it is suggested in the literature that the accumulation of a foreign protease inhibitor in plants may be compromised by the proteolytic activity of endogenous proteases. The inhibitor may also accumulate significantly in the plant but can be degraded by endogenous proteases at the time of extraction, as previously stated for the OC-1 cystatine (Michaud et al., 2000, in Michaud Ed., Austin Tex., pp. 195-200).

Considering that plants are an excellent source of nutritive proteins that are highly sensitive to proteolysis by endogenous proteases, it is highly desirable to develop an efficient method to preserve the protein content of plants and plant extracts during the process of food production, without altering the normal metabolism or development of the plant.

### DISCLOSURE OF INVENTION

One aim of the present invention is to provide a method for increasing the nutritive value of plant or portion thereof comprising without significantly altering the natural physiology of the plant or portion thereof, comprising neutralizing the activity or action of at least one plant proteolysis degradation on at least one endogenous proteins with an inhibitor released from the plant or plant extract at the time plant cells thereof are disrupted. The portion can be an extract or a concentrate of said plant. It is understood that the neutralizing can be partial or total depending on the needs.

The said plant cells are disrupted during processing of said plant or plant portion during preparation of an extract or a concentrate, or during swallowing or digestion of the plant or plant portion.

Also, the neutralization of proteolysis degradation is obtained by genetically altering the plant to cause condition for inhibiting totally or partially at least one proteolytic reaction specifically involved in the degradation of the endogenous proteins when cell disruption takes place.

The plant protease is inhibited not during the growth of the plant in order to preserve the activity of said plant protease during the growth and natural physiology of the plant.

The method of the present invention allows to increase the stability of the endogenous proteins during swallowing or digestion process in a human or an animal for a predetermined period of time. For example, the degradation and cell disruption of the plant portion that is swallowed and digested in an animal can be completed only when it reaches the stomach, or the intestines in order to deliver to the system whole protein or peptides having better nutritive effects.

The method can be performed to neutralize protease selected from the group consisting of cysteine proteases, aspartate proteases, metallo proteases, serine proteases, threonine proteases, and multispecific, broad range proteases.

Preferentially, the plant is genetically transformed with an expression cassette comprising a promoter operably linked with a factor or peptide causing the neutralizing conditions of the proteolytic degradation when the cells are lysed or disrupted. For example, the neutralizing factor can be linked to a leader peptide, a signal peptide or an anchorage peptide or a protein to lead or anchor the protease inhibitor to a cell part or extracellular compartment in manner to protect the endogenous proteins from the activity of a plant protease during the processing of the plant extracts. The cell part or extracellular compartment can be selected so as to protect the endogenous proteins from the activity of a plant protease at the time of cell disruption during the processing of the plant extracts but not during the growth of the plant in order to preserve the activity of the plant protease during the growth of the plant. For example, the cell part can be an organelle selected from the group consisting of a mitochondria, a chloroplast, a storage vacuole, an endoplasmic reticulum, and a cytosol.

In accordance with another aspect of the present invention, the method can be carried out using protease inhibitor selected from the group consisting of an antibody or a fragment thereof, a sens-mRNA or anti-sens mRNA, an inhibitor of trancription or a regulator thereof, a inhibitor of translation or a regulator thereof, an inhibitor of leading or signal peptide, an inhibitor of metabolic acquisition of activity of a protease, a protease-specific protease, and an affinity peptide protease leading to segregation to said protease into an organelle or a cell compartment. The cassette may comprised of an expression vector in which a coding sequence is regulated by a constitutive, a bipolar, or an inducible promoter. The expression vector may also comprised of an inducible promoter that is a tissue-specific promoter, a time-specific promoter, or a wound inducible promoter.

Preferentially, the plant or plant cells on which the method of the present invention is realized are from an alfalfa or a potato.

One object of the present invention is to provide a method for increasing the nutritive value of a plant extracts by preventing degradation of endogenous proteins at the time of cell disruption, during the processing of a plant, plant tissues, plant portions, plant cells or plant extracts.

A further object of the present invention is to provide a method wherein the prevention of endogenous protein degradation occurs in plant and plant cells by neutralizing protease-mediated proteolysis of the said endogenous protein at the time the cells are disrupted, lysed, swallowed or digested.

The subject invention also concerns plants and plant tissues that are capable of expressing high levels of stable proteins which are localized, for example but not limited to, as protein bodies within the plant cell.

Another object of the present invention is to provide a method wherein neutralization of proteolysis comprises genetically altering the functional properties of a protease, said protease including alfalfa and potato proteases.

A further object of the present invention is to provide a plant or plant cell preserving yield of plant endogenous protein at the time of cell disruption. It will be recognized additionally by someone skilled in the art that cell disruption may occur when a plant is grinded, for example, or process in the preparation of a plant extract or a plant concentrate. Cell disruption may also occur when the plant or plant cells are chewed or swallowed during eating. This is performed by inhibiting protease-mediated proteolysis of the said endogenous protein. Preferentially, the plant includes Medicago sativa (alfalfa) and potato.

For the purpose of the present invention, the following terms are defined below.

The term “endogenous protein” is intended to mean a protein that is naturally produced by the plant or plant cell.

The terms “promoter” or “promoter region” or “transcriptional regulatory sequence” as used herein mean a DNA sequence, usually found upstream (5′) to a coding sequence, that controls expression of the coding sequence by controlling production of messenger RNA (mRNA) by providing the recognition site for RNA polymerase and/or other factors necessary for initiation of transcription at the correct site. As contemplated herein, a promoter or promoter region includes variations of promoters derived by means of ligation to various regulatory sequences, random or controlled mutagenesis, and addition or duplication of enhancer sequences. The promoter region disclosed herein, and biologically functional equivalents thereof, are responsible for driving the transcription of gene sequences under their control when introduced into a host as part of a suitable recombinant vector, as demonstrated by its ability to produce mRNA.

The expressions “plant cell” or “plant part” as used herein are intended to refer to or include plantlets, protoplasts, calli, roots, tubers, propagules, seeds, seedlings, pollen, any other plant tissues.

The term “protease” is intended to mean an enzyme that performs directly or indirectly the degradation of polypeptides into smaller peptides, fragments or amino acids, or into a form leading to the loss of the stability of a protein of interest.

### BRIEF DESCRIPTION OF THE DRAWINGS

FIG. 1 illustrates the time-course of alfalfa (A) or potato (B) leaf protein degradation by endogenous proteases in crude extracts, at pH 4.5 and pH 7.5.

FIG. 2 illustrates the inhibition of Rubisco-Bodipy-FL degradation by alfalfa (cultivar Saranak) leaf proteases by usual concentrations of serine-type PIs (A) or cysteine-type PIs (B).

FIG. 3 illustrates the inhibition of Rubisco-Bodipy-FL degradation by potato (cultivar Kennebec) leaf proteases by usual concentrations of serine-type P is (A) or aspartate and cysteine-type PIs (B).

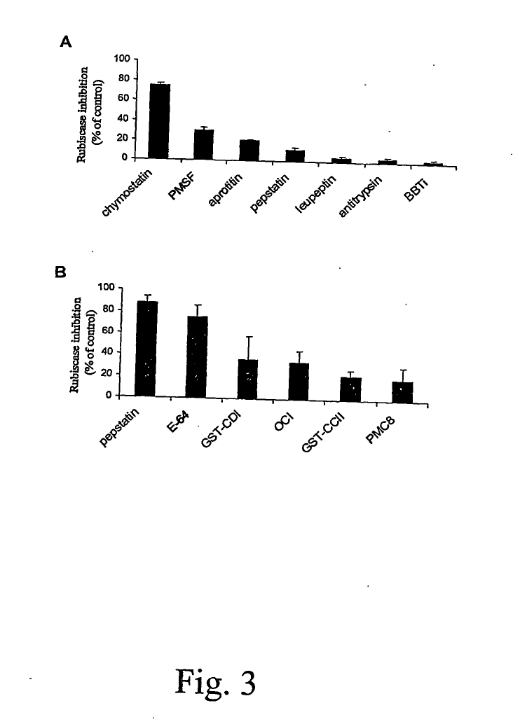

FIG. 4 illustrates the rubiscase activity measured in transgenic potato plant expressing low (+), high (++) or very high (+++) levels of cdi mRNA.

### MODES OF CARRYING OUT THE INVENTION

The present invention provides methods for increasing the endogenous protein content plant extracts by expressing protease inhibitors in those plants to be processed for animal feeding or used in agriculture, industry and medicine.

The present invention proposes a novel approach to improve forage crop protein quality by genetically introducing selected protease inhibitors to prevent the proteolysis of endogenous plant proteins.

Also, the present invention is directed to a method for producing plant lines genetically altered to inhibit at least one protease for preserving the integrity of the endogenous protein of interest at the time of cell disruption during the processing of plant extracts.

In one aspect of the present invention, strategies to specifically express and address the protease inhibitor are chosen not to affect negatively the metabolism or development of the transgenic plant.

In one embodiment of the invention, a protease inhibitor can be targeted to a different subcellular compartment from the natural localization of a targeted protease in order to preserve the vital activity of the protease during growth of the plant, and promote protection of endogenous proteins at the time of cell disruption during the processing of plant extracts.

The present invention can also be realized through inhibition of the production or synthesis of a protease in the plant of interest.

Alternatively, inhibition of the activity of a protease in a plant or plant tissue can be carried out by modulating the transcription or translation mechanisms in a way to prevent the protease from acquiring its activity or activity potential.

In another preferred embodiment of the invention strategies to specifically express and target the protease inhibitor are chosen to significantly not to affect or preserve the metabolism or development of the plant. It will be understood here that the normal physiology of a plant or plant cell in which conditions for inhibiting the activity or action of a protease at the time of cell lysis, the protein of interest, is preferentially not altered. For example, but not limited to, a plant in which genetic modification results in inhibition of a protease therein, will grow at the same rate than a non modified plant. Under another aspect, the protein synthesis is also not altered by the conditions in the plant or plant cell resulting in the inhibition of a protease.

In accordance with the present invention, there is provided a method that will give conditions causing the inhibition, partial or total, of the action or the activity of the proteases at the time the plant cells are disrupted or lysed. Preferentially, the method makes use of protease inhibitors, and use of sequences to genetically engineer plants or plant cells in a manner to protect from the activity of a protease the endogenous proteins produced in these plants or plant cells. Another condition of inhibiting the activity of a protease according to the present invention is that the inhibitor binds directly the protein of interest to avoid the protease to access the cleavage site for example, of binds directly the protease in order to block its action or activity.

Alternatively, the protease inhibition according to the invention, can be performed in changing the specificity of the protease itself or the condition that cause changes in the specificity of the protease for a nutritive protein of interest during cell lysis. The specificity changing of the protease will preferentially not affect its activity naturally occurring in a plant or plant cell.

In accordance with the present invention, there is provided a method for improving the nutritive value of plants or plant extracts by enhancing the yield of their protein content. More particularly, the nutritive value is improved by preventing the degradation of the endogenous proteins catalyzed by plant proteases.

One embodiment of the present invention is to provide a method for increasing the forage quality of a plant comprising transforming a plant or plant tissue with a polynucleotide molecule that encodes for at least one protease inhibitor, or a protein that will cause conditions neutralizing the action or the activity of a protease at the time the cells are disrupted or lysed.

In another embodiment of the present invention, there is provided a method for increasing the recovery yield of endogenous nutritive proteins in plants or plant cells, the method comprising the step of obtaining plants or plants cells expressing one or more inhibitor(s) which targets the endogenous plant proteases implicated in the degradation of endogenous proteins.

In a broad sense, the method may consist of a) targeting a protease inhibitor in a particular subcellular compartment or b) targeting two protease inhibitors into separate subcellular compartments or c) targeting two protease inhibitors in a same subcellular compartment. More than two protease inhibitors may also be expressed in one or several subcellular compartments. Subcellular compartments may be chosen from the group comprising cytosol, endoplasmic reticulum, extracellular compartment, mitochondria, chloroplasts, apoplast and storage vacuole.

The choice of the targeting strategy for the protease inhibitor is critical as it may affect dramatically the metabolism or development of the transgenic plant by altering vital functions of endogenous proteases. For example, the pea vicillin, a vacuolar protein can be expressed in the endoplasmic reticulum of the alfalfa cell by using a retention signal (Wandelt et al., 1992, Plant J. 2:181-192).

Other examples of the targeting of foreign proteins into mitochondria and chloroplast are described in the art as well as examples of vacuolar targeting of foreign proteins in plants (Di Sansebastiano et al., 1998, Plant J. 15:449-457).

Removal of a targeting peptide from a protein can result in a cytosolic protein, and a secretory peptide added to a foreign protein will promote the secretion of the protein. The natural secretory peptide of a foreign protein may be correctly processed by the plant secretory machinery.

According to one embodiment, the present invention can be performed by using plants or plant cells comprising a DNA sequence encoding an inhibitor operably linked to a promoter and which optionally comprises the fusion of a targeting peptide to address the inhibitor to a particular subcellular or extracellular compartment of the plant or plant cells.

Alternatively, the invention may be realized in plants or plant cells obtained by the crossing-over of two separate plants comprising both a particular protease inhibitor operably linked to a particular promoter and which optionally comprises the fusion of a particular targeting peptide to address the inhibitor to a particular subcellular or extracellular compartment of the plant or plant cells.

Any gene encoding a potent protease inhibitor may be introduced into the genome of a plant to reduce the proteolytic activity in the plant which is desirable for increasing the plant endogenous protein content for animal feeding. Examples of protease inhibitors that can be recombinantly produced into alfalfa or potato consist of but are not limited to the plant cystatins OCI, OCII and TMC-8 to inhibit the plant's cysteine protease and the human serpin alpha-1-anti-chymotrypsin (AACT) to inhibit the plant's serine protease. An example of protease inhibitor that can be expressed in a potato plant is an aspartate-type protease inhibitor (CD-I), to inhibit rhe plant's aspartate proteases.

The invention may also use other types of protease inhibitors that could be chosen in, but are not limited to, the group of antibodies or fragments of antibodies specific to a protease or a protease propeptide.

According to one aspect of the present invention, an antibody or a fragment thereof specific to a protease that hinders normal activity of the enzyme can be produced in a transgenic plant. This method of inhibition depends on the capacity of the antibody to bind its antigen in the plant cell. Hence, it is required, that the plant can be capable to produce an antibody or a fragment thereof, and this can be achieved by genetically transforming the plant with the transgene or transgenes needed to produce a complete and active antibody. Different antibodies can be expressed in transgenic plants, including immunoglobulins (IgG, IgA and IgM), single chain antibody fragments (ScFv), fragment antigen binding domain (Fab), and heavy chain variable domains.

In one aspect of the present invention, an antibody or a fragment thereof can be targeted to a different subcellular compartment from the natural localization of a targeted protease in order to preserve the vital activity of the protease during the growth of the plant, and promote protection of endogenous proteins at the time of extraction and plant food production. In one particular embodiment of the present invention, the proteolysis is partially or totally prevented by producing genetically modified plant or plant cells. The protein degradation by proteases inhibition can be specifically allowed in the genetically modified plants or plant cells produced throughout the processes of plant harvesting plant stocking, or even swallowing or digestion in a human or an animal.

As illustrated herein, to perform the method of the present invention, a plant can be genetically modified to express at least one recombinant protease inhibitor directed essentially against serine proteases, cysteine proteases, aspartate proteases, threonine proteases and metalloproteases.

In another embodiment of the present invention, two protease inhibitor genes can be co-inserted in the plant genome, in the same sub-cellular compartment or not as described herein.

Different strategies can be employed to engineer plants. For example, this can be carried out, without limiting it thereto, by inserting a protease inhibitor encoding gene into the genome of a plant

Also, the present invention can be carried out by using a constitutive or an inducible promoter to control the expression of a protease inhibitor. For example, the expression of the inhibitor could be induced at the time of harvesting only, by the addition of an inductive agent prior to harvesting. A plant expressing one or several protease inhibitors may be crossed-over with a plant expressing one or more protease inhibitors.

One embodiment of this invention proposes the use of various promoters to control the expression of protease inhibitors. Promoters that could be used in the proposed method are, without being limited to: constitutive, inducible, viral, synthetic, development-specific, tissue-specific, temporally regulated, spatially regulated, and spatio-temporally regulated.

The promoters that can be used in performing the present invention may be inducible in response to the presence or absence of an exogenous inducer and thereby enable to express the inhibitor at the same time as the protein thereby reduce the degradation of said protein by the plant proteases during the whole production process. Inducible promoters to be used according to the present invention may be chosen from the group of, but are not limited to: wound-inducible promoters, nutrient-inducible promoters, cold-inducible promoters. For example, but without limiting it thereto, the inducible promoters of the present invention may include the nitrite reductase promoter derived from the genome of Medicaco sativa (alfalfa) (WO 01/25454). A heavy metal-inducible promoter can also be used, such as the 35S CaMV-derived promoter which is known to control expression of beta-glucuronidase in tobacco in presence of Cd2+ (Brandle et al., 1993, Genome 36:255-260). Another example of inducible promoter is a low temperature-inducible promoter which naturally controls the expression of the cor15a gene of Arabidopsis thaliana (Dordrecht, 1994, Plant Mol. Biol. 24: 701-713).

According to one embodiment of the present invention, an expression vector designed to be inserted into a plant or plant cell to inhibit the activity of a protease, may comprise two DNA sequences encoding for two protease inhibitors. The two encoding DNA sequences can be generally operably linked by a unique bipolar promoter.

In relation with the present invention, different transformation methods may be used to insert protease inhibitor genes into the genome of various plants. This includes particle bombardment, tissue electroporation, microinjection, direct DNA transfer to protoplasts using polyethylene glycol (PEG) and finally the silicon carbide “whisker” method. All those methods are known from the art.

Plants contemplated within the scope of the invention include forage crop plants, including, for example, alfalfa, clover, corn silage, sorghum and other leguminous crops, transformed to express the proteins of the invention.

Also contemplated within the scope of the present invention are plants for human or animal consumption which have been transformed to express proteins or RNAs that enhance the protein quality of the plant for improved nutrition.

One plant that can be useful in performing the present invention is the alfalfa, Medicago sativa. Alfalfa is considered to be the most important cultivated forage crop in the world and is often referred to as “Queen of the forage crops” because it is widely grown, has a superb balance of vitamins and minerals, is high yielding, is an excellent source of biological nitrogen fixation, and it serves as an attractive nectar source for honeybees. Alfalfa has been bred for years for both forage quality and plant performance.

The stability and integrity of different proteins in a forage plant, plant tissue or plant extract of high nutritive value can be suitably preserved through the application of the present invention. For example, but without limiting it thereto, rubisco is one of such proteins conferring high suitable nutritive value to a plant

### EXAMPLES

The present invention will be more readily understood by referring to the following examples that are given to illustrate the invention rather than to limit its scope.

### Example I

### Degradation and Protection of Proteins in Potato and Alfalfa Leaf Extracts

To establish a rationale for the identification of target proteases in plant extracts and the choice of inhibitors effective against these proteases, proteolytic activities in leaf tissues of alfalfa and potato were monitored using as a substrate ribulose 1,5-biphosphate carboxylase/oxygenase (Rubisco), the most abundant protein in plants.

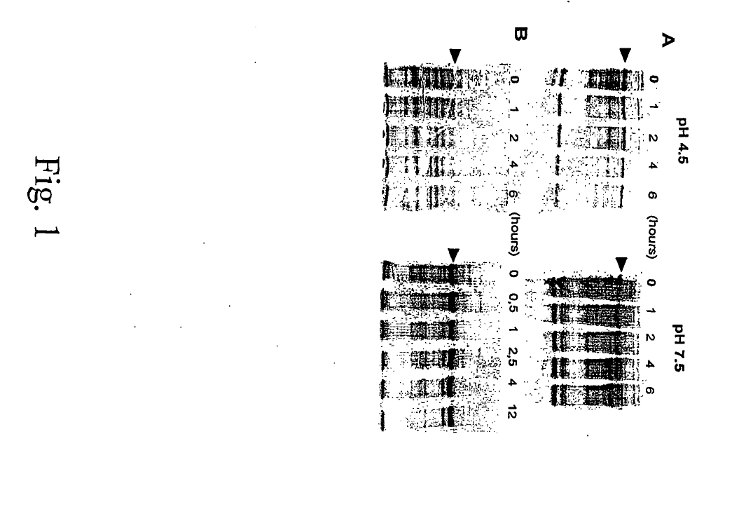

FIG. 1 illustrates the fate of endogenous proteins in alfalfa (A) and potato (B) leaf extracts, showing their limited stability after extraction at low pH. Leaf samples (first to fourth leaves from the apex) were ground in liquid nitrogen. Proteins were extracted (1:3 w/v) in 50 mM Tris-HCl (pH 7.5) or 0.1 M citrate phosphate (pH 4.5), in the presence of 10 mM β-mercaptoethanol. The soluble protein extracts were agitated for 10 min at 4° C., and centrifuged for 10 min at 18000 g. The supernatants were recovered and protein concentrations were determined with the Bradford's method (Bradford, 1976, Anal Biochem. 72:248-254). Proteins in Tris-HCl buffer were adjusted to a final concentration of 1 mg/ml, whereas proteins in citrate phosphate buffer were adjusted to a final concentration of 0.5 mg/ml. The proteolysis assay was performed by incubating the extracts at 25° C. Aliquots of 70 μl were pipetted at different times after initiation of the reaction, immediately mixed with 30 μl of denaturing electrophoresis buffer (200 mM Tris-HCl pH 8.8, 10% (v/v) β-mercaptoethanol, 2% (w/v) SDS, 10% (v/v) glycerol), and heated at 95° C. for 5 min. Seven μg of the denatured proteins were then loaded on a standard 10% SDS-PAGE gel and stained by Coomassie. FIG. 1 shows that a significant fraction of the large, 52-kDa subunit of Rubisco (arrow), for instance, was degraded by endogenous proteases at pH 4.0 and pH 7.5, after only a few hours.

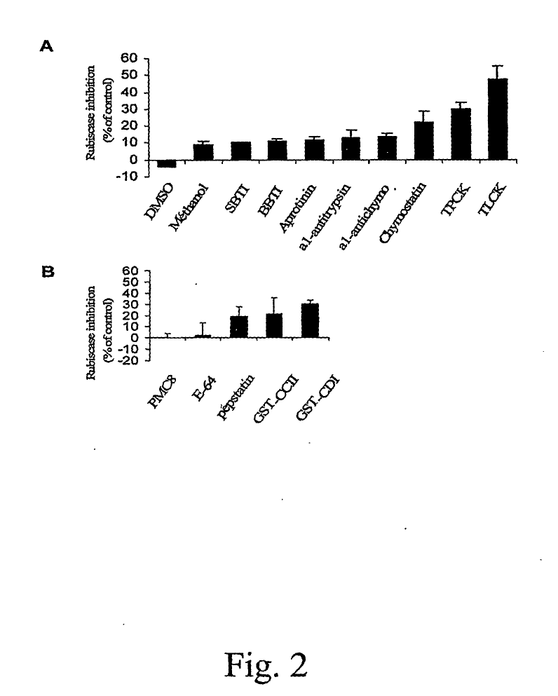

To monitor the impact of diagnostic PIs and plant recombinant PIs on protease activity of potato leaf and alfalfa extracts, the total protease activity against Rubisco (named rubiscase activity) was determined by fluorometry, using Rubisco conjugated with the succinyl ester fluorophore, Bodipy® FL,SE (Molecular Probes, Oreg., USA) as a substrate. Conjugation of Rubisco to Bodipy® FL,SE was done following the supplier's instructions. Plant tissues were ground in liquid nitrogen and extracted (1:3 w/v) in a 100 mM Hepes (pH 7.5) buffer containing 2 mM MgCl₂, 1 mM DTT and 1% (w/v) PVPP. The soluble extract was centrifuged at 12000 rpm for 15 min at 4° C. The supernatant was removed and dialyzed on a Sephadex G-25 column pre-equilibrated with reaction buffer consisting either of 50 mM Hepes (pH 7.5) (neutral reaction buffer), or 0.15 M potassium acetate (pH 5.5) (acidic reaction buffer). Fifty μL of the plant extracts were pre-incubated for 20 min. at 20° C. with 5 μL reaction buffer (control), 5 μL DMSO, 5 μL methanol, 5 μL SBTI (1 mg/ml), 5 μL BBTI (1 mg/ml) 5 μL aprotinin (1 mM), 5 μL α-1 antitrypsin (1 mg/ml), 5 μL α-1 antichymotrypsin (1 mg/ml), 5 μL chymostatin (6 mM), 5 μL TPCK (20 mg/ml), 5 μL TLCK (20 mg/ml), 5 μL PMC8, 5 μL E-64 (100 mM), 5 μL pepstatin (1 mM), 5 μL GST-CCII or 5 μL GST-CDI. All diagnostic PIs were obtained from Sigma-Aldrich (St-Louis, USA). GST-CDI and GST-CCII were expressed as recombinant proteins in Escherichia coli and purified on an affinity glutathione column (Brunelle et al., 1999, Arch. Insect Biochem. Physiol. 42:88-98). Fluorometric assays were conducted at 30° C. with Rubisco-Bodily-FL as substrate. Two fig of Rubisco-Bodipy-FL was added to the reaction mixture and the volume was completed to 100 μl with neutral (assays with DMSO, methanol, SBTI, BBTI, aprotinin, antitrypsin, antichymotrypsin, chymostatin, PMSF, TPCK, TLCK) or acidic (assays with PMC8, E-64, pepstatin, GST-CCII, GST-CDI) reaction buffers. Fluorescence intensity was measured 100 times over a 5,000-sec period using a Fluostar Polastar Galaxy fluorimeter (BMG Lab Technologies), with excitation and emission filters of 485 nm and 520 nm, respectively. Protease activity, expressed in units of fluorescence per sec, corresponded to the slope of the emission curve. As shown on FIGS. 2 (alfalfa) and 3 (potato), Rubisco hydrolysis by both alfalfa and potato leaf proteases was significantly delayed when diagnostic proteinase inhibitors were added, pointing out certain groups of protease as interesting targets for the development of strategies aimed at protecting endogenous nutritive protein integrity via inhibition of the plant endogenous proteases. These data also show that the inhibition of a single proteases (or protease groups) is sufficient to protect a significant part of the proteins present in crude extracts, despite the presence of other (insensitive) proteases in the medium. Inhibitors of chymotrypsin (TPCK, chymostatin, α1-antichymotrypsin), trypsin (TLCK, □1-antitrypsin), cysteine (CCII) and aspartate (pepstatin, CDI) proteases, in particular, showed interesting protective effects, causing rubiscase activity rate decreases ranging from ˜15 to 40%.

### Example II

### Effect of the Ectopic Expression of a Cathepsin D Proteinase Inhibitor into Potato on the Stability of an Endogenous Protein (e.g. Rubisco)

To assess the impact of ectopically expressing a recombinant protease inhibitor in the plant on the activity of endogenous proteases during extraction (ex vitro), a cathepsin D inhibitor from tomato, tomato CDI (Werner et al. 1993, Plant Physiol. 103:1473), was integrated into an expression vector and stably expressed into potato (cultivar Kennebec), under the control of the cauliflower mosaic virus 35S (CaMV 35S) promoter (CD lines). The tomato CDI-encoding DNA sequence was isolated from the expression vector pGEX-3X/CDI (Brunelle et al. 1999, Arch. Insect Biochem. Physiol. 42:88-98) by digestion with BamHI and EcoRI, and subcloned between the BamHI and EcoRI cloning sites of the commercial vector pCambia 2300 (CAMBIA, Canberra, Australia). The CaMV 35S promoter was isolated from the commercial plasmid pBI-121 (Clontech, Palo Alto, Calif.) using a BamHI/SalI treatment, and then ligated between the BamHI and SalI cloning sites of the pCambia construct including the cdi transgene. Transgenic controls (SPCD lines) expressing the selection marker neomycine phosphotransferase (NPTii) but no CDI were also devised by integrating the cdi transgene with no promoter. Axenically-grown plantlets of potato (Solanum tuberosum L. cultivar Kennebec) were used as source material for genetic transformation. The plantlets were maintained on MS multiplication medium (Murashige and Skoog 1962, Physiol. Plant 15:473-497) supplemented with 0.8% (w/v) agar (Difco, Detroit, Mich.) and 3% (w/v) sucrose, in a tissue culture room at 22° C. under a light intensity of 60 μmol·m⁻²·s⁻¹ and a 16 h/day photoperiod provided by cool fluorescent lights. Leaf discs about 10 mm in diameter were genetically-transformed using the bacterial vector Agrobacterium tumefaciens LBA4404 as described by Wenzler et al. (1989, Plant Sci. 63:79-85), except that cefotaxime, instead of carbenicillin, was used for A. tumefaciens growth control. Regenerated shoots were transferred onto a selection medium with kanamycin and cefotaxime, for root regeneration and plantlet multiplication. For acclimation, the plantlets were transferred for 14 days in a growth chamber under a 24°/21° C. day/night temperature cycle, a 12-h L:D photoperiod, a light intensity of 200 μmol·m⁻²·s⁻¹ and a relative humidity of 60%, before being transferred in a greenhouse under standard growth conditions. Integration of the nptii (marker) transgene in kanamycin-resistant plants was confirmed by PCR, using DNA extracted from the fourth, fifth and sixth leaves (from the apex) of ˜30-cm potato plants, according to Edwards et al. (1991, Nuc. Acids Res. 19:1349). Expression of the cdi transgene in transgenic lines was monitored by RT-PCR and northern blotting, using total RNA extracted from the fourth, fifth and sixth leaves of nptii transgene-positive plants, as described by Logemann et al. (1987, Anal Biochem. 163:16-20).

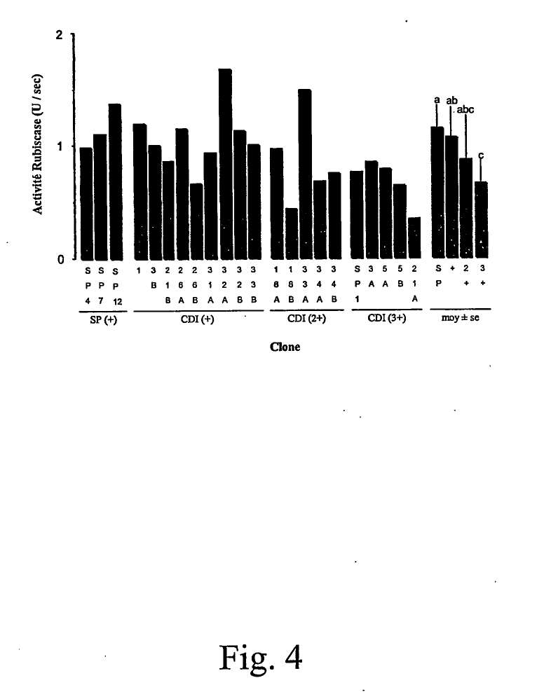

To monitor the impact of the ectopic expression of CDI in potato on endogenous protease activity, the total protease activity against Rubisco was determined by the fluorometric method using Rubisco-Bodipy-FL as a substrate (see above). Soluble proteins were extracted in 0.15 M potassium acetate (pH 5.5) (1:3 w/v) from the leaves (fourth leaves from apex) of control (SPCD lines) or cdi-expressing (CD lines) transgenic potato plants. Fifty μL of soluble protein extracts were incubated at 30° C. with 2 μg of Rubisco-Bodipy (in 100 μl extraction buffer) in the presence of 20 mM L-cysteine. As shown in FIG. 4, Rubisco-Bodipy-FL degradation by leaf protein extracts from transgenic lines expressing high levels of recombinant cdi mRNA (e.g., clone 21A) was significantly lowered as compared to the control (Fisher's LSD test (p<0.05). More precisely, the inhibition rate of rubiscase activity reached ˜40% under the conditions of the assay, compared to transgenic controls with no CDI (SP4, SP7 and SP12), clearly showing a reduced “proteolytic power” against Rubisco for the CDI-expressing lines.

Sequestration of CDI into the cytoplasm of potato leaf cells did not affect significantly the growth and development of the transgenics, suggesting that the putative target aspartate proteases were not significantly or negatively affected by the inhibitor in vivo. On the other hand, the data presented in FIG. 4 show that the tomato recombinant CDI may quickly inhibit protein hydrolysis by endogenous proteases during harvesting, homogenization and/or extraction procedures, resulting in significant protection of the major leaf protein, Rubisco.

### Example III

### Protection of Endogenous Proteins by the Ectopic Expression of Protease Inhibitors in Alfalfa

As described above, alfalfa leaf cells contain a considerable amount of poorly-specific proteases released in the medium during extraction. Similarly, Wandelt and co-workers (1992, Plant J. 2:181-192) showed that vicilin, a vacuolar seed storage protein from Vicia faba, is accumulated in large amounts in alfalfa leaf cells when the peptide signaling scheme directing its usual accumulation into the vacuole is impaired, clearly showing the potential negative effect of this plant's vacuolar proteases on the stability of heterologous proteins expressed in leaves; It is now generally assumed that an important source of non-specific proteolytic activities in plant leaf cells is accounted for by proteases active in the acidic-to-mildly acidic pH range, usually belonging to the cysteine and aspartate classes of proteolytic enzymes (Callis, 1995, Plant Cell 7:845-857). It is obvious from our data presented above that different types of proteases—for instance chymostatin-sensitive proteases—may also have a significant impact on the stability of useful endogenous proteins (see FIG. 2). As poorly-specific proteases are often found in cell compartments other than the cytoplasm, PIs active against these proteases (e.g. α1-antichymotrypsin or CCII) may be expressed in the cytoplasmic compartment of leaf cells without interfering negatively with the plant proteases in vivo, but then be ready to act against these same enzymes after cell breakage during the recovery process.

In practice, the strategies that may be used consist in developing transgenic lines of forage plants—e.g., alfalfa—expressing an appropriate PI, and then using this line to produce animal feeding conserving a higher protein content after harvest, by protection of endogenous protein from endogenous proteases.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for increasing the nutritive value of plant or portion thereof comprising without significantly altering the natural physiology of said plant or portion thereof, comprising neutralizing the activity or action of at least one plant proteolysis degradation on at least one endogenous proteins with an inhibitor released from said plant or plant extract at the time plant cells thereof are disrupted.

2. The method of claim 1, wherein said portion is an extract or a concentrate of said plant.

3. The method of claim 1, wherein said neutralizing is partial or total.

4. The method of claim 1, wherein said plant cells are disrupted during processing of said plant or plant portion during preparation of an extract or a concentrate, or during swallowing or digestion of said plant or plant portion.

5. The method of claim 1, wherein said neutralization of proteolysis degradation is obtained by genetically altering said plant to cause condition for inhibiting totally or partially at least one proteolytic reaction specifically involved in the degradation of the endogenous proteins when cell disruption takes place.

6. The method of claim 5, wherein said proteolytic reaction is performed by a protease.

7. The method of claim 6, wherein said plant protease is inhibited when said cell disruption takes place during said processing of said plant or plant extract but not during growth of the plant in order to preserve the activity of said plant protease during the growth and natural physiology of the plant.

8. The method of claim 1, which increases stability of said endogenous proteins during swallowing or digestion process in a human or an animal for a predetermined period of time.

9. The method of claim 1, wherein said neutralization is performed on a protease selected from the group consisting of cysteine proteases, aspartate proteases, metallo proteases, serine proteases, threonine proteases, and multispecific, broad range proteases.

10. The method of claim 1, wherein said plant is transformed with an expression cassette comprising a promoter operably linked with a factor or peptide causing said neutralization of proteolytic degradation.

11. The method of claim 10, comprising linking said factor to a leader peptide, a signal peptide or an anchorage peptide or a protein to lead or anchor said protease inhibitor to a cell part or extracellular compartment in manner to protect said endogenous proteins from the activity of a plant protease during the processing of said plant extracts.

12. The method of claim 10, wherein said factor is a protease inhibitor.

13. The method of claim 11, wherein the cell part or extracellular compartment is selected so as to protect said endogenous proteins from the activity of a plant protease at the time of cell disruption during the processing of said plant extracts but not during the growth of the plant in order to preserve the activity of said plant protease during the growth of the plant.

14. The method of claim 13, wherein said cell part is an organelle selected from the group consisting of a mitochondria, a chloroplast, a storage vacuole, an endoplasmic reticulum, and a cytosol.

15. The method of claim 12, wherein said protease inhibitor is selected from the group consisting of an antibody or a fragment thereof, a sens-mRNA or anti-sens mRNA, an inhibitor of trancription or a regulator thereof, a inhibitor of translation or a regulator thereof, an inhibitor of leading or signal peptide, an inhibitor of metabolic acquisition of activity of a protease, a protease-specific protease, and an affinity peptide protease leading to segregation to said protease into an organelle or a cell compartment.

16. The method of claim 1, wherein said plant is an alfalfa or a potato.

17. The method of claim 10, wherein said cassette comprises an expression vector in which a coding sequence is regulated by a constitutive, a bipolar, or an inducible promoter.

18. The method of claim 17, wherein said inducible promoter is a tissue-specific promoter, a time-specific promoter, or a wound inducible promoter.
